(19) United States
(12) Patent Application Publication
(10) Pub. No.: US 2009/0312402 A1
(43) Pub. Date: Dec. 17, 2009

(54) ENCAPSULATED NANOPARTICLES FOR DRUG DELIVERY

(76) Inventors: Christopher H. Contag, Stanford, CA (US); Rajesh R. Shinde, Stanford, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/469,578

(22) Filed: May 20, 2009

Related U.S. Application Data
(60) Provisional application No. 61/128,364, filed on May 20, 2008.

(51) Int. Cl. A61K 48/00

(52) U.S. Cl. 514/44 R; 977/773

(57) ABSTRACT

Compositions and methods are provided for preparing nanosized biologically nucleic acids, including agents formulated for target specific drug delivery. In some embodiments, the nanoparticles comprise a polymer coating, which can provide for controlled delivery, targeting, controlled release, and the like. In other embodiments, the nanoparticles comprise a target specific tag for targeting the nanoparticles to a site of interest, e.g. tissue, cell, etc.

### GOVERNMENT RIGHTS

This invention was made with Government support under contract 0120999 awarded by the National Science Foundation. The Government has certain rights in this invention.

### BACKGROUND OF THE INVENTION

The physical and chemical factors that allow polynucleotides to perform their functions in the cell have been studied for several decades. Recent advances in the synthesis and manipulation of polynucleotides have allowed this field to move ahead especially rapidly during the past fifteen years. In particular, short oligonucleotides such as antisense, RNAi and shRNAs have been found to be effective in modulating gene expression, where the oligonucleotides may be native forms of nucleic acids, or may be synthetic analogs thereof. For example, small inhibitory RNAs (siRNA) and short hairpin RNAs (shRNA) can effectively target specific mRNA molecules for degradation in cells.

MicroRNAs (miRNAs) are an abundant class of non-coding RNAs that are believed to be important in many biological processes through regulation of gene expression. These ˜22-nt RNAs can repress the expression of protein-coding genes by targeting cognate messenger RNAs for degradation or translational repression. The mechanisms by which miRNAs exert these effects are unclear, as is whether they have any specific role in the adaptive immune response.

Small interfering RNA (siRNA) has emerged as a new class of potential drug candidates for a range of diseases due to their low toxicity, high specificity and efficiency. These nucleic acids work by degrading the messenger RNA (mRNA) expression via an antisense RNA sequence. In theory siRNA allow for targeting of any gene or multiple of interest by careful design of the anti-sense sequence. This approach is well placed for rapid development of drugs and can potentially reduce preclinical and clinical development.

Although the use of nucleic acids such as RNA inhibition (RNAi) has proven to be very useful for controlling gene expression in cell culture, in vivo applications have been limited due to problems of delivery to the target tissue. Consequently, RNAi's have a huge potential for controlling gene expression in vivo and for use as therapeutics if effective delivery tools were available.

The formulation of delivery systems that would serve to protect nucleic acids from degradation and enzymatic attack are of great interest. The present invention addresses this issue.

Liposomes and lipoplexes have been widely used as delivery vehicles for siRNA to target the liver (Morrisseyet al. (2005) Nature biotechnology 23, 1002-1007; Zimmermann et al. (2006) Nature 441, 111-114). Shyh-Dar Li et al. have targeted c-Myc and VEGF in lung cancer tumors using liposomal formulations (Li et al. (2006) Annals of the New York Academy of Sciences 1082, 1-81 Li et al. (2006) Molecular pharmaceutics 3, 579-588; Li et al. (2008) Mol Ther 16, 942-946; Li et al. (2008) J Control Release 126, 77-84). Polyconjugates are another approach to delivering siRNAs in vivo, and have shown some efficacy in knocking down ApoB in hepatocytes, though the small size of the polyconjugates may limit their applications due to rapid clearance. Cationic polymers are also currently being used as a material for formulating delivery vehicles for siRNAs wherein the polymers complex with the siRNA to form nanoparticles (Akinc et al. (2003) Bioconjugate chemistry 14, 979-988; Jon et al. (2003) Biomacromolecules 4, 1759-1762; Schiffelers, et al. (2004) Nucleic acids research 32, e149; Heidel et al. (2007) Proceedings of the National Academy of Sciences of the United States of America 104, 5715-5721; Zugates et al. (2007) Bioconjugate chemistry 18, 1887-1896). These formulations have shown some success in treating tumors, but the charged interactions between the phosphate group on the siRNA and the cationic group on the polymer tends to get disrupted in serum leading to reduced circulation times. The presence of charged moieties also leads to aggregation of the nanoparticles during formulation or after injection.

### SUMMARY OF THE INVENTION

Compositions and methods are provided for encapsulation of nucleic acids into nanoparticles. Nanoprecipitation is a process in which nanosized particles are formed by the precipitation of a material such as a polymer in a fluid in which it is not soluble. The fluid acts as a non-solvent for the polymer. The polymer is made up in a solvent that is miscible in this non-solvent, such that upon exposure of the polymer solution to the non-solvent, there is a movement of the solvent into the non-solvent phase. The polymer chains also collapse and precipitate in order to avoid contact with the non-solvent. Factors that can influence the size and encapsulation efficiencies of the nanoparticles, include, without limitation, the molecular weight of the polymer, the choice of solvent and non-solvent, the temperature, and various additives that can be added to the polymer solution and/or the non-solvent. In some embodiments, the nanoparticles comprise a polymer coating, which can provide for controlled delivery, targeting, controlled release, and the like. In other embodiments, the nanoparticles comprise a target specific tag for targeting the nanoparticles to a site of interest, e.g. tissue, cell, etc.

In some embodiments of the invention, the nucleic acid is titrated against a cationic amphipathic molecule, e.g. a cationic lipid such as 1,2-dioleoyi-sn-glycero-3-trimethylammonium-propane (DOTAP). The charged groups on the cationic amphipathic molecule nullify the negatively charged phosphate groups on the nucleic acid, thus converting it into a hydrophobic entity (hydrophobic ion-pair-HIP). The HIP is co-solubilized into solvents that also dissolve polymers of interest for encapsulation, e.g. PLA, PGA, PLGA, PLA-PEG, PLG-PEG, PLGA-PEG, etc. Solvents include, without limitation, dichloromethane (DCM), chloroform (CHF), tetrahydrofuran (THF), ethyl acetate, etc. Miscible non-solvents for nanoprecipitation include, without limitation, ethanol, methanol, butanol etc.

In some embodiments, the polymer for encapsulation is a conjugate of PLA, PLG and/or PLGA and PEG. The PEG moiety reduces RES uptake and presents a platform for attaching ligands and antibodies to enhance target specificity of the nanoparticles. Encapsulating the nucleic acid with a biodegradable polymer also helps prevent their degradation by nucleases. In related embodiments, an amine-rich polymer is inserted in such a conjugate between the PEG moiety and the PLA, PLG and/or PLGA moiety. Such an insertion does not interfere with nanoparticles uptake into cells, but contributes to endosomal escape into the cytoplasm, and thereby enhancing the transport of nucleic acids into cells. Amine rich polymers of interest include, without limitation, diethylone triamine (DETA), tetraethylene pentaamine (TEPA), ethylene diamine (EDA), tetraethylene tetraamine (TETA), bis(hexamethylene triamine) (BHMT) and pentaethylene hexamine (PEHA).

### BRIEF DESCRIPTION OF THE DRAWINGS

FIG. 1. Schematic of nanoparticles synthesis.

FIG. 2. RLG transgenic mouse: β-actin promoter-Loxp-Renilla Luciferase-Stop-Loxp-Click Beetle Luciferase-Monster GFP; Alb.Cre transgenic mouse: Albumin promoter-Cre.

FIG. 3. Graph of particle size.

FIG. 4. Release of siRNA from nanoparticles.

FIG. 5A-B. A. Gel of siRNA released from nanoparticles. B. Inhibition of luciferase activity.

FIG. 6. Decrease in luciferase levels.

FIGS. 7A-7C. A-B. In vivo imaging. C. Fractionated fluorescence levels.

FIG. 8. Fractional bioluminescence knockdown.

FIG. 9. PEG conjugates.

FIG. 10. Nanoparticle uptake in HepG2 cells with the use of tetraethylene pentaamine (TEPA) coating.

### DETAILED DESCRIPTION OF THE EMBODIMENTS

For preparing nanoparticles of nucleic acid with a polymer, a nanoprecipitation method is used where the nucleic acid is neutralized by titration with a cationic amphipathic molecule to create a HIP. The HIP and polymer for encapsulation are dissolved in an organic solvent, e.g. CHF, etc. to form a homogenous solution. The polymer concentration can range from 1 mg/ml to 100 mg/ml, and preferably at least about 10 mg/ml and not more than about 30 mg/ml. The RNAi-HIP concentration can also be varied to change the encapsulation ratio, e.g. from about 100:1 polymer:HIP (by weight) to about 1000:1, to about 10:1, 1:1, etc. The homogenous solution is added drop wise or injected into a mildly polar non-solvent, where the volume of non-solvent may be varied, e.g. from about 1:1 solvent:non-solvent; from about 1:2; 2:1, 10:1, 1:10; 1:100, 100:1, etc.

It has been shown that nanoparticles are taken up by macrophages and cleared by the reticular endothelial system (RES). The clearance can be reduced significantly by attaching nonionic hydrophilic polymers such as poly (ethylene glycol) on the particle surface. The nanoprecipitation technique described above is amenable for obtaining nanoparticles with a hydrophilic PEG coating. In order to achieve such a coating, the biodegradable polymer, PLA, PGA or PLGA, is optionally coupled to PEG. Since the PEG is soluble in the solvent as well as the non-solvent, when the solvent starts to move into the non-solvent, the PEG also starts to orient in the same direction. Since the PEG is tethered to the hydrophobic portion, such as PLA, it cannot move into the solution and dissolve. Thus, the particles that are formed have a predominance of PEG on the surface. If the free end of PEG has a functional group, e.g. —OH, —NH₂, —COOH, —SH, etc., it can be used as a point of attachment for ligands, e.g. sugar such as galactose, antibodies, DNA, RNA, peptides or any other moiety that is used for specific targeting.

In related embodiments, an amine-rich polymer is inserted in such a conjugate between the PEG moiety and the PLA, PLG and/or PLGA moiety. Such an insertion does not interfere with nanoparticles uptake into cells, but contributes to endosomal escape in cells, and thereby unexpectedly increasing the bioavailable nucleic acid. Preferably such amine rich polymers have a pKa of 6.0 or lower. Amine rich polymers of interest generally comprise at least two, at least three, at least four, at least five or more amine groups, which may be present on a hydrocarbon scaffold. Polymers of interest include diethylene triamine (DETA), tetraethylene pentaamine (TEPA), ethylene diamine (EDA), tetraethylene tetraamine (TETA), bis(hexamethylene triamine) (BHMT) and pentaethylene hexamine (PEHA). Natural and unnatural amino acids as polyamines can also be used as amine-rich moieties that can be inserted between the PEG moiety and PLA, PLG and/or PLGA moiety.

In an alternative embodiment, a nucleic acid is neutralized by titration with a cationic amphipathic molecule to create a HIP. The HIP and polymer for encapsulation are dissolved in an organic solvent, e.g. CHF, etc. to form a homogenous solution. The polymer concentration can range from 1 mg/ml to 100 mg/ml, and preferably at least about 10 mg/ml and not more than about 30 mg/ml. The RNAi-HIP concentration can also be varied to change the encapsulation ratio, e.g. from about 100:1 polymer:HIP (by weight) to about 1000:1, to about 10:1, 1:1, etc. The homogenous solution is added drop wise or injected into a non-miscible non-solvent. Generally the organic solvent w/ the siRNA-HIP and polymer is emulsified to form an emulsion in a non-solvent (water), and this emulsified solution mix is then further homogenized to form fine emulsion droplets. This fine emulsion is then subjected to reduced vacuum to remove the organic solvent. Upon removal of the organic solvent, the polymer solidifies to form nanoparticles w/ siRNA encapsulated inside, where the volume of non-solvent may be varied, e.g. from about 1:1 solvent:non-solvent; from about 1:2; 2:1, 10:1, 1:10; 1:100, 100:1, etc.

The methods of the invention allow encapsulation of at least about 50%, at least about 80%, at least about 90% or more of the initial nucleic acid into the nanoparticles, and this can be further increased by varying the composition and precipitation conditions. Achieving high encapsulation efficiencies is important since the nucleic acids are expensive. Substantially all of the encapsulated nucleic acid can be released within about one week, although the rate of release can be enhanced or delayed by using different polymers, additives and/or different initial compositions. The nanoparticles are in the size range of about 50 nm to about 500 nm, with most of them in the sub-200 nm range.

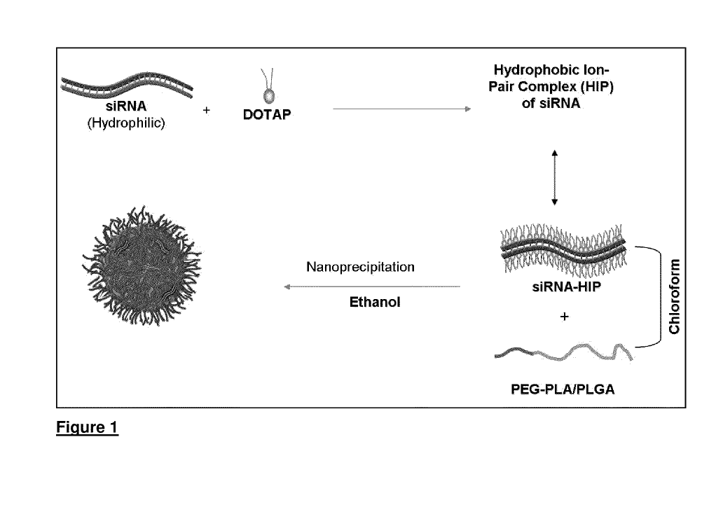

The zeta potential for nanoparticles of the invention may range from at least about +5 mV to about −5 mV. The nanoparticle does not comprise a lipid bilayer, but is usually a solid core having a PEG outer coat, as shown in FIG. 1.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The methods of the invention find particular use with active agents, e.g. nucleic acids, that have a short half-life in vivo due to degradation. As used herein, the term “genetic agent” refers to polynucleotides and analogs thereof. Genetic agents such as DNA can result in an introduced change in the genetic composition of a cell, e.g. through the integration of the sequence into a chromosome. Genetic agents such as antisense or siRNA oligonucleotides can also affect the expression of proteins without changing the cell's genotype, by interfering with the transcription or translation of mRNA. The effect of a genetic agent is to increase or decrease expression of one or more gene products in the cell.

A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al (1998) Science 282, 744-746. cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

In one embodiment, the genetic agent is an antisense or siRNA sequence that acts to reduce expression of the targeted sequence. Antisense or siRNA nucleic acids are designed to specifically bind to RNA, resulting in the formation of RNA-DNA or RNA-RNA hybrids, with an arrest of DNA replication, reverse transcription or messenger RNA translation. Gene expression is reduced through various mechanisms. Antisense nucleic acids based on a selected nucleic acid sequence can interfere with expression of the corresponding gene.

Antisense oligonucleotides (ODN), include synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Among nucleic acid oligonucleotides are included phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Nucleic acid molecules of interest also include nucleic acid conjugates. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed.

Also of interest are RNAi agents. RNAi agents are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

dsRNA can be prepared according to any of a number of methods that are known in the art, including in vitro and in vivo methods, as well as by synthetic chemistry approaches. Examples of such methods include, but are not limited to, the methods described by Sadher et al., (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed., Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In some embodiments, the nucleic acid is treated prior to the encapsulation process to increase the hydrophobicity, e.g. by treatment with a cationic amphipathic molecule, e.g. 1,2-Dioleoyl-3-Trimethylammonium-Propane (Chloride Salt) (DOTAP), prior to solubilizing with the polymer. For example the BLIGH DYER technique may be used for making the nucleic acid hydrophobic.

Amphiphilic molecules have a hydrophilic head group and a hydrophobic tail group, where the hydrophobic group and hydrophilic group are joined by a covalent bond, or by a variable length linker group. The linker portion may be a bifunctional aliphatic compounds which can include heteroatoms or bifunctional aromatic compounds. Preferred linker portions include, e.g. variable length polyethylene glycol, polypropylene glycol, polyglycine, bifunctional aliphatic compounds, for example amino caproic acid, or bifunctional aromatic compounds.

Amphipathic molecules of interest include lipids, which group includes fatty acids, neutral fats such as triacylglycerols, fatty acid esters and soaps, long chain (fatty) alcohols and waxes, sphingoids and other long chain bases, glycolipids, sphingolipids, carotenes, polyprenols, sterols, and the like, as well as terpenes and isoprenoids. For example, molecules such as diacetylene phospholipids may find use as neutral amphipathic molecules.

Cationic amphipathic molecules form tight complexes with the nucleic acid, thereby condensing it and protecting it from nuclease degradation. In addition, polycationic nanoparticles may act to mediate transfection by improving association with negatively-charged cellular membranes by giving the complexes a positive charge; masking the nucleic acid from neutralizing antibodies or opsonins which are in circulation; increasing systemic circulation time by reduction of non-specific clearance mechanisms in the body, i.e. macrophages, etc.; decreasing immunogenicity; and/or enhancing transport from the cytoplasm to the nucleus where DNA may be transcribed.

The term “cationic amphipathic molecules” is intended to encompass molecules that are positively charged at physiological pH, and more particularly, constitutively positively charged molecules, comprising, for example, a quaternary ammonium salt moiety. Cationic amphipathic molecules used for gene delivery typically consist of a hydrophilic polar head group and lipophilic aliphatic chains. Similarly, cholesterol derivatives having a cationic polar head group may also be useful. See, for example, Farhood et al., (1992) Biochim. Biophys. Acta 1111:239-246; Vigneron et al. (1996) Proc. Natl. Acad. Sci. (USA) 93:9682-9686.

Cationic amphipathic molecules of interest include, for example, imidazolinium derivatives (WO 95/14380), guanidine derivatives (WO 95/14381), phosphatidyl choline derivatives (WO 95/35301), and piperazine derivatives (WO 95/14651). Examples of cationic lipids that may be used in the present invention include DOTIM (also called BODAI) (Solodin et al., (1995) Biochem. 34: 13537-13544), DDAB (Rose et al., (1991) BioTechniques 10(4):520-525), DOTMA (U.S. Pat. No. 5,550,289), DOTAP (Eibl and Wooley (1979) Biophys. Chem. 10:261-271), DMRIE (Feigner et al., (1994) J. Biol. Chem. 269(4): 2550-2561), EDMPC (commercially available from Avanti Polar Lipids, Alabaster, Ala.), DCChol (Gau and Huang (1991) Biochem. Biophys. Res. Comm. 179:280-285), DOGS (Behr et al., (1989) Proc. Natl. Acad. Sci. USA, 86:6982-6986), MBOP (also called MeBOP) (WO 95/14651), and those described in WO 97/00241. In addition, nanoparticles having more than one cationic species may be used to produce complexes according to the method of the present invention.

In some embodiments, the nucleic acid and the polymer are soluble in a single entity, as described above where the nucleic acid is neutralized with a cationic amphipathic molecule. The concentration of the nucleic acid and the polymer in the solvent, as well as the nucleic acid/polymer ratio, allows for control of particle size and encapsulation yield. The concentrations are selected to provide for the desired end product by optimization, as is known in the art. In general, a lower concentration of nucleic acid is selected for smaller particle sizes, and a higher concentration for larger particle sizes. A higher ratio of polymer to nucleic acid will provide for a thicker polymer encapsulation, while a lower ratio of polymer to nucleic acid will provide for a thinner coating. The concentration of nucleic acid will usually be at least about 0.001 mg/ml, more usually at least about 0.01 mg/ml, at least about 0.1 mg/ml, or 1 mg/ml, and not more than about 100 mg/ml, usually not more than about 10 mg/ml. The concentration of polymer will usually be at least about 0.01 mg/ml, more usually at least about 0.1 mg/ml, at least about 1 mg/ml, and not more than about 100 mg/ml, usually not more than about 50 mg/ml. The ratio of compound to polymer as a weight percent will vary, from around about 1:1000; 1:500; 1:100, 1:50; 1:10; 1:5, and the like.

Solvents of interest are organic solvents, including, without limitation, dichloromethane (DCM), chloroform (CHF), tetrahydrofuran (THF), ethyl acetate, etc. The solvent solution with nucleic acid and polymer is dropped or injected at a set flow rate into a vessel filled with a miscible non-solvent. Flow rate may be optimized for each nucleic acid/polymer/solvent system. The selection is based on the desired yield and particle size. Miscible non-solvents for nanoprecipitation include, without limitation, ethanol, methanol, butanol etc.

Generally a temperature selected to maintain the stability of the nucleic acid polymer, and is usually not more than about 100° C., more usually not more than about 80° C., and may be not more than about 40° C., 30° C., or 20° C. When a polymer is included it is desirable to keep the temperature below the glass transition temperature of the polymer, which typically ranges from 45-65° C., e.g. for PLGA. Therefore in some embodiments a temperature of around about 40° C. is used to advantage.

The nanoparticle core may be covered with a substantially uniform coating, where the coating may be any biologically compatible polymer. Some examples of biodegradable polymers useful in the present invention include hydroxyaliphatic carboxylic acids, either homo- or copolymers, such as poly(lactic acid), poly(glycolic acid), Poly(dl-lactide/glycolide, poly(ethylene glycol); polysaccharides, e.g. lectins, glycosaminoglycans, e.g. chitosan; celluloses, acrylate polymers, and the like. The selection of coating may be determined by the desired rate of degradation after administration, by targeting to a desired tissue, e.g. in the use of lectins, by protection from oxidation, and the like.

In certain embodiments, a PEG moiety is conjugated to the encapsulation polymer, as described in the Examples. Chemical groups that find use in linking a targeting moiety to an amphipathic molecule also include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

The targeting moiety may be joined to PEG through a homo- or heterobifunctional linker having a group at one end capable of forming a stable linkage to the hydrophilic head group, and a group at the opposite end capable of forming a stable linkage to the targeting moiety. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N,N′-(1,3-phenylene)bismaleimide; N,N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodimide, bromo-tris(pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

A targeting moiety, as used herein, refers to all molecules capable of specifically binding to a particular target molecule and forming a bound complex. Thus the ligand and its corresponding target molecule form a specific binding pair.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

Examples of targeting moieties include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting moieties of particular interest include peptidomimetics, peptides, antibodies and antibody fragments (e.g. the Fab′ fragment). For example, β-D-lactose has been attached on the surface to target the asiologlycoprotein (ASG) found in liver cells which are in contact with the circulating blood pool.

Cellular targets include tissue specific cell surface molecules, for targeting to specific sites of interest, e.g. neural cells, liver cells, bone marrow cells, kidney cells, pancreatic cells, muscle cells, and the like. For example, nanoparticles targeted to hematopoietic stem cells may comprise targeting moieties specific for CD34, ligands for c-kit, etc. Nanoparticles targeted to lymphocytic cells may comprise targeting moieties specific for a variety of well known and characterized markers, e.g. B220, Thy-1, and the like.

### Pharmaceutical Compositions

The process of fabricating the nanoparticles involves the conversion of polynucleic acid entities into hydrophobic entities by using cationic materials such as lipids, polyethyleneimines (PEI), polyamino acids, polyvinyl pyrrolidone (PVP), cationic lipids, etc. The nucleic acids are carefully titrated against these cationic materials to make them hydrophobic, wherein the nucleic acid is dissolved into an aqueous phase, and the DOTAP is dissolved into an organic solvent. The two solutions are mixed with an alcohol such as methanol or ethanol in the ratio of 1:2.1:1 (siRNA:methanol:DOTAP). The siRNA and DOTAP are used at equimolar concentrations. This leads to the formation of a monophase, which upon mixing for some time, 15 minutes to more than 3 hours, leads to the formation of the HIP complex. Upon nullification of the charges, the entire entity becomes hydrophobic with the long carbon chains of the lipid allowing for greater solubility in organic solvents. After mixing, the monophase is separated into an organic phase and an aqueous phase by addition of equal volumes of the organic solvent and water or buffer. The HIP can then be extracted from the organic phase and either dried or used as is.

The HIP is then solubilized into an appropriate organic solvent such as chloroform along with the polymer. For example, polymer at concentrations ranging from 10-100 mg/ml is dissolved in chloroform along with the HIP. The HIP concentration can be varied from 0 to 10% of the total polymeric concentration. The solution is homogenous and added dropwise or injected into a bath of non-solvents, i.e. solvents that precipitate the polymer. Where the polymer is coated to PEG, the free end of PEG can carry different functional groups as mentioned above, thus providing a platform for surface modification of the nanoparticles with ligands, antibodies, aptamers etc.

The organic solvents in this mixture may then evaporated using either rotary evaporator at not more than 40° C., or by stirring for long periods of time. The nanoparticles are recovered in the water phase, which is then centrifuged to recover the nanoparticle pellet. The nanoparticles can also be recovered using various filtration setups with appropriate membrane cutoffs.

The nanoparticles of the invention may be incorporated in a pharmaceutical formulation. Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel; sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration. The effective amount of a therapeutic composition to be given to a particular patient will depend on a variety of factors, several of which will be different from patient to patient. A competent clinician will be able to determine an effective amount of a therapeutic agent. The compositions can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, antibody moieties that do not provoke immune responses are preferred.

Those of skill will readily appreciate that dose levels can vary as a function of the specific compound, the severity of the symptoms and the susceptibility of the subject to side effects. Some of the specific complexes are more potent than others. Preferred dosages for a given agent are readily determinable by those of skill in the art by a variety of means. A preferred means is to measure the physiological potency of a given compound.

### Experimental

### PLGA-Based siRNA-Nanocarrier for Targeted Delivery: β-Actin Driven Click Beetle Luciferase as a Model Target in Liver

siRNA has evolved into a potentially new class of drugs to administer gene therapy for a wide range of illnesses, including cancer and cardiovascular diseases. One of the key challenges in achieving this goal is the lack of an appropriate delivery vehicle. Nanocarriers are a potentially powerful platform for delivering a variety of drugs and have been pursued as a vehicle of choice for siRNA. Hydrophobic biodegradable polymers such as poly(lactic-co-glycolic acid) (PLGA) find use as materials for encapsulating siRNA for targeted delivery. The nanoparticles were coated with polyethylene glycol (PEG) to reduce immunogenicity and allow for attachment of targeting moieties such as ligands, antibodies or aptamers. These nanoparticles were successfully delivered to the liver to knockdown the reporter gene, click beetle luciferase, driven by a β-actin promoter, in transgenic mice. The results establish the use of these materials for siRNA delivery systemically. Our approach can remove the need for a double-emulsion technique to make nanoparticles by making particles using nanoprecipitation.

Alternatively, this approach can be effectively used in the emulsion-diffusion method, wherein the siRNA and the hydrophobic polymer are solubilized into hydrophobic organic solvents such as dichloromethane, chloroform, ethyl acetate. This solution can then be emulsified into an aqueous solvent such as water with the use of appropriate stabilizers and surfactants, and then the organic solvent can be evaporated under reduced pressure.

This was accomplished by enhancing the lipophilicity of siRNA using appropriate cationic materials such as 1,2-dioleoyl-3-Trimethylammonium-Propane (DOTAP). With a pKa of around 4.5, DOTAP also served as a buffer to protect the siRNA in the acidic PLGA microenvironment. These nanoparticles were successfully utilized to knockdown the luciferase signal in the mouse liver by greater than 60% with a single injection containing siRNA at 2.5 mg/kg.

We decided to develop a delivery vehicle that utilizes FDA approved materials thus reducing the timescales for development and a simple fabrication process that could be scalable. The approach was modular, thus providing increased adaptability towards different formulations. We used PLGA, which is a biodegradable hydrophobic polymer that has been approved for medical use for over three decades, and has shown potential use as materials for delivery of chemotherapeutic agents. Attachment of PEG at one end of PLGA allows for fabrication of nanoparticles using the nanoprecipitation approach which results in a hydrophobic core (PLGA) and a hydrophilic surface (PEG) (see Gref et al. (1995) Advanced Drug Delivery Reviews 16, 215-233). Other fabrication processes such as the double-emulsion method, (Remaut et al. (2007) Materials Science and Engineering 58, 117-161) are known in the art.

In order to allow for a homogenous solution of PLGA and the nucleic acid payload, the siRNA was made hydrophobic using cationic materials such as 1,2-dioleoyloxy-3-trimethylammoniumpropane chloride (DOTAP). This change in the lipophilicity of the nucleic acid allowed for fabrication of nanoparticles using standard nanoprecipitation techniques. Using PEG-PLGA block copolymers allowed for nanoparticles with appropriate functional groups on the surface, as shown in FIG. 1.

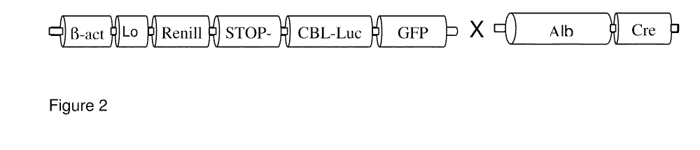

The nanoparticles were characterized for size, zeta potential, release of siRNA and stability. Gene knockdown in cells and animals was investigated using Click Beetle Luciferase (CBL) as a model reporter, wherein siRNA sequences were designed to target the luciferase and delivery was quantified as a decrease in bioluminescence signal Liver was chosen as the target organ for these studies to demonstrate the feasibility of our delivery vehicle. For in-vivo studies, a transgenic mouse was developed that expressed CBL exclusively in hepatocytes. This was achieved by using the CRE-LOX system, wherein an albumin-CRE mouse was bred against a β-actin-lox-RLuc-Stop-lox-CBL mouse with a CMV enhancer (FIG. 2). siRNA targeting the CBL was delivered to the liver using the nanoparticles with galactose (Gal) as a targeting ligand, and the efficacy was measured using bioluminescence imaging (BLI).

### Results and Discussion

Nanoparticle Fabrication and Characterization. Hydrophobic Ion-Pair (HIP) complex of tRNA and cationic lipids such as DOTAP were formed using the Bligh-Dyer monophase which were then extracted into the organic phase containing dichloromethane or chloroform Reimer et al. (1995) Biochemistry 34, 12877-12883; Wong et al. (1996) Biochemistry 35, 5756-5763). Molar ratios of at least 1:1 were necessary to obtain greater than 95% extraction of the nucleic acids from the aqueous phase into the organic phase.

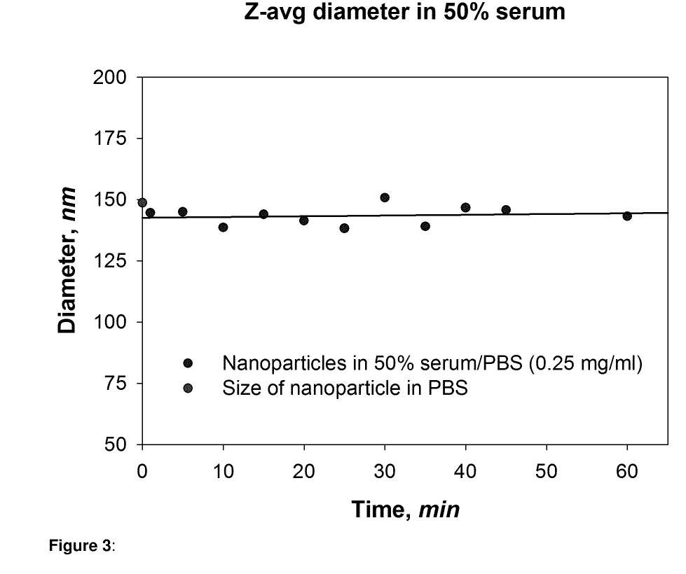

siRNA-HIP and PEG-PLGA were dissolved in chloroform at various concentrations and nanoparticles were formed by drop-wise addition to an ethanol bath with constant stirring. The process yielded consistently high encapsulation efficiencies >74%. Increasing molecular weight of PLGA increased the encapsulation from 74% to 88%, whereas changing the concentration of the polymer did not significantly affect the encapsulation efficiency. The maximum encapsulation efficiency that was obtained was >98%. The nanoparticles were also characterized for their surface charge by determining the zeta potential, and the presence of PEG on the surface helped to reduce the zeta potential of PLGA from −25 to less than −5. The zeta potentials of the nanoparticles were within the desirable range of −5 to +5 which is appropriate for reducing non-specific adsorption by proteins in the serum. The diameter of the nanoparticles was less than 200 nm which is considered as an appropriate cutoff for targeting tumor vasculature. FIG. 3 represents the diameter of a nanoparticle, containing siRNA, placed in a 50% serum/PBS media for 1 hour. The presence of PEG prevents agglomeration of the nanoparticles as evidenced by the constant diameter over 1 hour.

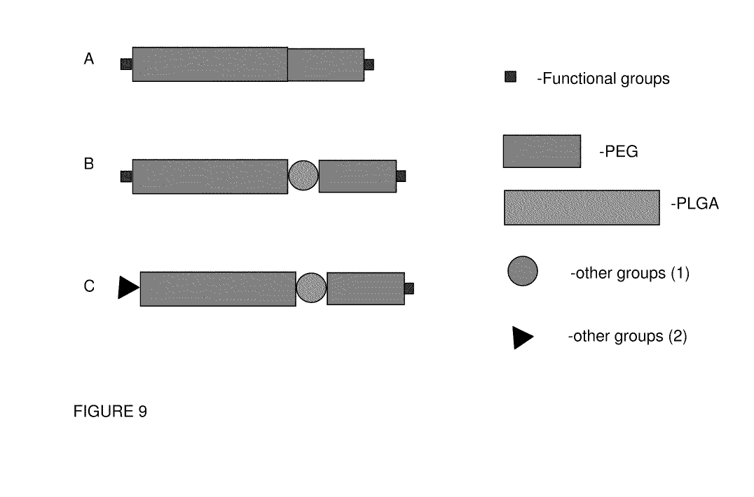

The ratio of ethanol to chloroform was varied to determine its effect on encapsulation, and there was no noticeable difference in the final nanoparticle composition. Based on these experiments, all subsequent nanoparticles were obtained using polymer concentrations of 10 mg/ml in chloroform and ethanol to chloroform volume ratios of 1-2. The nanoparticles can be stored for long periods of time as dry formulations using appropriate lyoprotectants such as sucrose (Cheng et al (2007) Biomaterials 28, 869-876) or PVA/sucrose/mannitol (Wendorf et al. (2006) Journal of pharmaceutical sciences 95, 2738-2750). The nanoparticles did not exhibit any significant change in the size upon reconstitution into either pure water or buffer (FIG. 9)

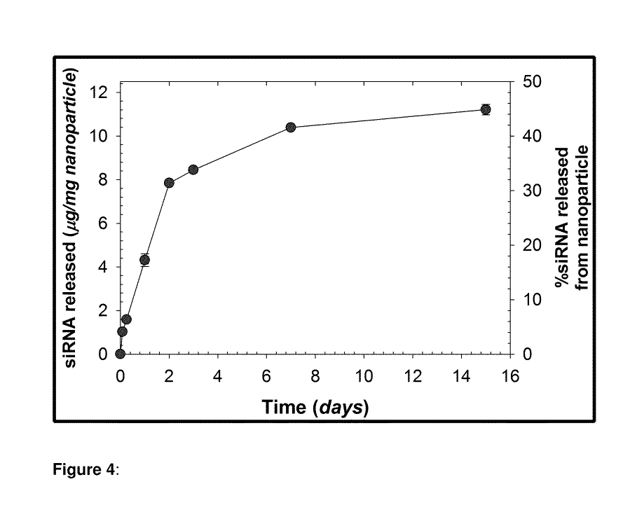

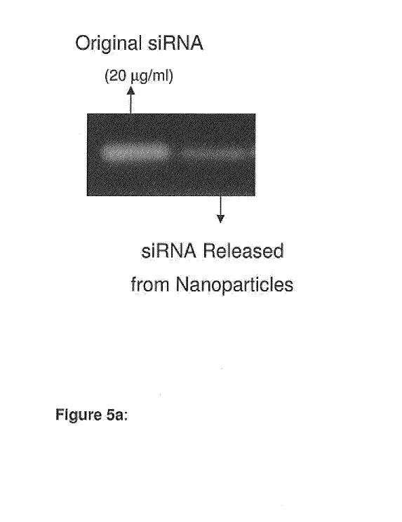

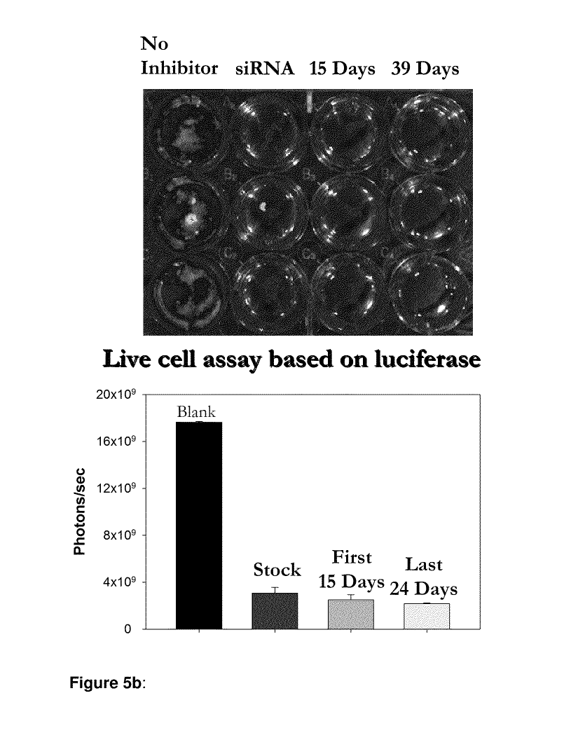

siRNA Release from Nanoparticles. The nanoparticles were characterized for release of siRNA in PBS at 37° C. The siRNA was quantified using Sybr Gold® (Invitrogen) which allowed for determining μg levels in the solution. The nanoparticles released ˜40-60% of the siRNA payload over a period of 4 weeks, with a majority of release occurring in 48 hours. A representative rate of release is shown in FIG. 4. This result is an improvement over other systems developed for DNA (Wang et al. (1999) J Control Release 57, 9-18; Hirosue et al (2001) J Control Release 70, 231-242). The absolute amount of siRNA released reaches 10 μg/(mg nanoparticle) over a one week period. The siRNA that was released was also tested for any loss of integrity or activity (FIG. 5). siRNA nanoparticles were incubated in PBS for 15 days, and 24 days. siRNA was removed from the PBS during this time and tested for degradation by gel electrophoresis (FIG. 5a). There was no visible difference in the size of the original siRNA and siRNA released from the nanoparticles. The siRNA were also tested for activity against CBL in HepG2 cells using Lipofectamine RNAiMax® (Invitrogen). HepG2 cells were stably transfected with CBL drive by a CMV promoter (HepG2-CBL), and maintained under puromycin antibiotic selection. The results show no decrease in siRNA activity against CBL, as observed in FIG. 5b.

Liver cells such as hepatocytes and Kupffer cells express asioglycoprotein receptors (AsGPr) that accept ligands including galactose. Conjugation of galactose to the surface of nanoparticles should enhance the uptake by the liver (see Lim et al (2000) Bioconjugate chemistry 11, 688-695; Popielarski et al. (2005) Bioconjugate chemistry 16, 1071-1080; Popielarski et al. (2005) Bioconjugate chemistry 16, 1063-1070). The modular approach we have used allows for conjugating galactose to the free end of PEG, with PLGA attached to the other end. Upon formation of the nanoparticles, the galactose localizes to the surface of the nanoparticles. The nanoparticles were tested for the presence of these galactose ligands using the Amplex® Red Galactose Assay (invitrogen). Nanoparticles without galactose groups were used as control. Since the galactose was located on a spherical surface, the particles were incubated for more than 2 hours to obtain the total concentration. The assay measured galactose concentrations at 4.1 μg/mg nanoparticles, which was comparable to the theoretical value of 4 μg/mg nanoparticles. The results also demonstrate that the ligands were accessible to galactose oxidase for enzymatic activity, which is crucial when using galactose as a targeting moiety.

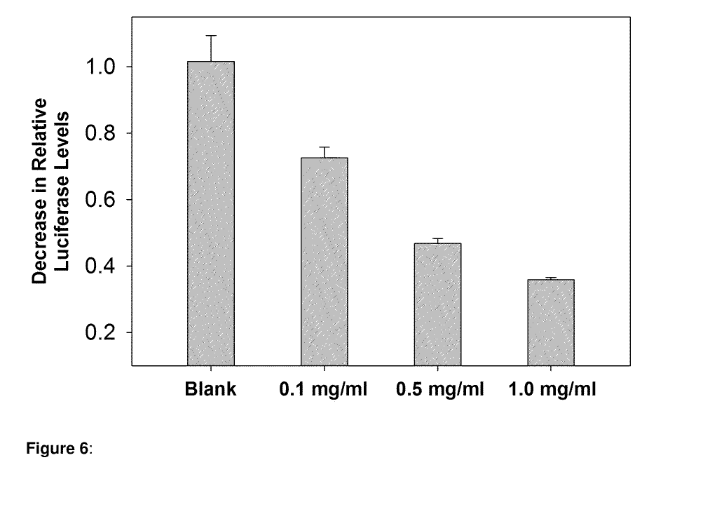

The nanoparticles were also tested for efficacy in knockdown of CBL in HepG2-CBL cell lines. Even though these vehicles are not optimized for cellular delivery, due to the presence of PEG and slower release extending over 48 hours, we observed a more than 60% knockdown of gene expression. FIG. 6 shows the results of CBL knockdown in HepG2-CBL cells with a dose dependent increase in efficacy up to nanoparticle concentrations of 1 mg/ml. The results demonstrate that the nanoparticles carrying the siRNA can deliver and knockdown gene expression in cells which is quite impressive considering that the rapid growth of the cells needs to be balanced with the release of the siRNA from the vehicle. Control experiments with mock siRNA did not show any knockdown of CBL expression.

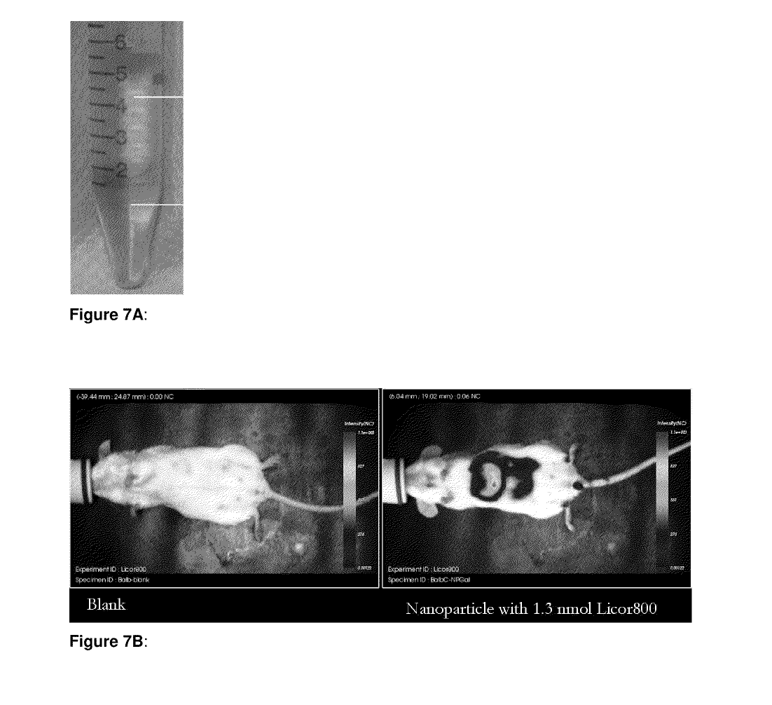

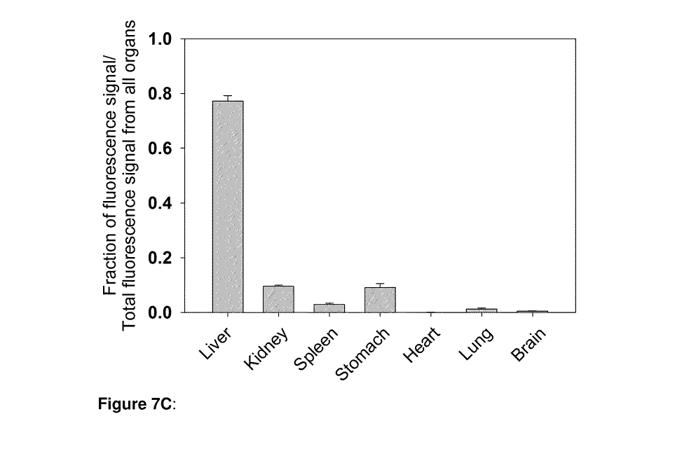

The biodistribution of the nanoparticles was investigated using NIR dyes that were conjugated to double-stranded DNA molecules. DOTAP was used to generate a HIP complex of these oligos, and extracted into chloroform as shown by the cyan color of the organic phase in FIG. 7a. The nanoparticles were then injected into Balb/C mice intravenously and imaged after 20 hours using the ART eXploreOptix fluorescence imaging system. The mouse on the left panel in FIG. 7B shows a representative background autofluorescence at 800 nm whereas the mouse on the right shows the fluorescence emitted by the Licor 800CW dye at 800 nm. The mice were excited at 745 nm. The organs were harvested from the animals and then imaged at the same excitation and emission wavelengths. The results of the fractional fluorescence from each organ are displayed in FIG. 7C and have been normalized for autofluorescence from control mice that without any Licor carrying nanoparticles. The nanoparticles appear to localize predominantly in the liver due to the presence of galactose ligands on the surface. The hepatocytes and kupffer cells have receptors that recognize and take up nanoparticles with galactose.

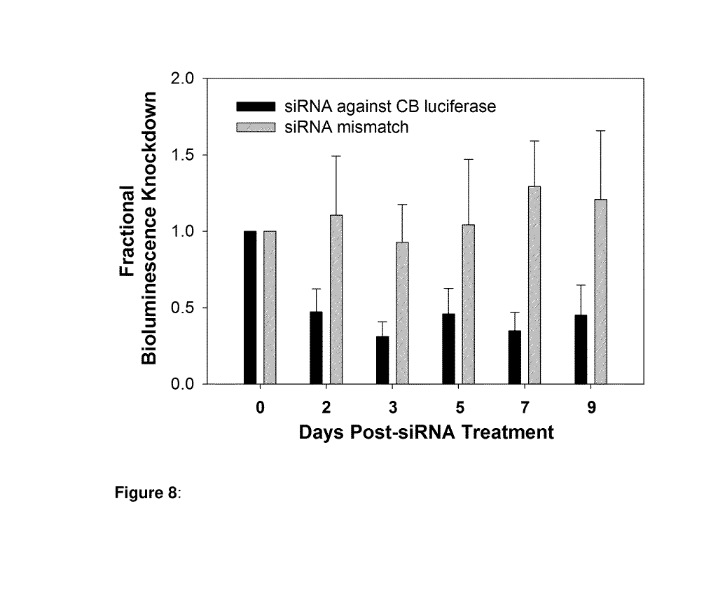

The efficacy of PLGA-based siRNA delivery vehicles was investigated by studying the knockdown of CBL expression in the liver of transgenic β-actin CBL mice. SiRNA at a concentration of 2.5 mg/kg mouse was introduced intravenously into the mice and the BLI was measured over a period of 7 days. The BLI signals from the liver was measured a day before the injections and were considered as the initial readout of CBL expression. BLI signals after treatment with siRNA were then normalized to these initial readouts and plotted as a measure of siRNA delivery efficacy to the liver, as shown in FIG. 8. We observe a reduction of greater than 60% in BLI signal from the liver after a single dose of siRNA. targeted towards CBL. In comparison, the control siRNA did not exhibit any reduction of BLI signal from the liver, as shown in FIG. 8.

### Methods:

Preparation of Copolymers of PLGA-PEG. the Synthesis of the Block Copolymers of PLGA-PEG was accomplished as described in previous studies (see Cheng et al. (2007) Biomaterials 28, 869-876). In brief, a PLGA polymer with a carboxylic acid terminating end group was used and coupled to PEG that was capped with amines. A coupling reaction between the amine and carboxylic acid groups was achieved using 1-ethyl-3-[3-dimethylaminopropyl]carbodiimide hydrochloride (EDC) and N-hydroxysuccinimide (NHS). First, the PLGA was activated with NHS in the presence of EDC in dichloromethane for at least 12 hours, and then precipitated into cold anhydrous ether. It was then washed a couple of times in a mixture of cold methanol and ether and dried in vacuum. The activated PLGA-NHS was reacted with H₃C-PEG-NH₂ in the presence of N—N-diisopropylethylamine (DIEA) in dichloromethane for more than 12 hours and the product was precipitated in cold methanol or ethanol. PEG was used at a 20-30% molar excess to PLGA. The copolymer was washed multiple times in cold methanol or ethanol to remove impurities.

This modular approach allows for development of different types of copolymers as indicated in FIG. 9. The red box could be PLGA, PLA, PGA, other hydrophobic biodegradable polymers such as poly orthoesters (POE) (Wang et al. (2004) Nature Materials 3, 190-196), poly caprolactone (PCL), polyanhydrides, polyhydroxyacids, polyesters, polyamides, polyphosphazenes etc. The green box can be polyethylene glycol (PEG), polysaccharides, poly vinyl pyrrolidones, poly vinylalcohol, poly(propylacrylic acids), synthetic peptides etc. that can shield the nanoparticles from serum proteins and provide a platform for attaching ligands. The small blue box can be any functional group such as —COOH, —OH, —NH₂, —SH, malemide, etc. It could also be a non-reactive group such as —CH₃ etc. The modular approach of this technique allows us to develop block copolymers with different functionalities. For example, we can attach the PEG onto the PLGA via a linker such that the PEG can be shed off under appropriate conditions (Romberg et al. (2008) Pharmaceutical Research 25, 55-7). Such an arrangement can allow for removal of the PEG polymer near the disease site under variations of pH, or inside the endosomes, or intracellularly. Another advantage of the modular approach is that we can insert a cationic entity such as a dendrimer, polyamines, polylysines etc. between the PEG and PLGA polymers.

In one such example, a polyamine tetra ethylene penta amine, (TEPA), CAS # 112-57-2, was introduced between a PLGA molecule and a PEG molecule. The reaction was achieved as follows. In brief a PLGA polymer with a carboxylic acid terminating end group was coupled to the TEPA. A coupling reaction between the TEPA and carboxylic acid groups was achieved using 1-ethyl-3-[3-dimethylaminopropyl]carbodiimide hydrochloride (EDC) and N-hydroxysuccinimide (NHS). First, the PLGA was activated with NHS in the presence of EDC in dichloromethane for at least 12 hours, and then precipitated into cold anhydrous ether. It was then washed a couple of times in a mixture of cold methanol and ether and dried in vacuum. The activated PLGA-NHS was reacted with a 2 to 50-fold molar excess of TEPA in the presence of N—N-diisopropylethylamine (DIEA) for more than 12 hours, and the product was precipitated in a mixture of cold anhydrous ether and methanol The TEPA-conjugated PLGA was washed at least 3 times in cold methanol or ethanol to remove the unreacted TEPA and other contaminants. The PLGA-TEPA was then used to attach PEG. In order to achieve PEG coupling to the PLGA-TEPA, a PEG with at least one —COOH end group was utilized. In this example, a COOH-PEG(5K)-CH3 polymer was used. The PLGA-TEPA and COOH-PEG(5K)-CH3 were dissolved in dichloromethane at a PEG molar excess of at least 1.2. EDC and NHS was added at the appropriate amounts (at least 5-fold molar excess to the reactants), and the reaction was carried out in the presence of DIEA for at least 12 hours. The final product was precipitated in cold anhydrous ether, and washed a few times in methanol or ethanol to yield PLGA-TEPA-PEG.

Nanoparticle Fabrication. The process of fabricating the nanoparticles involves the conversion of polynucleic acid entities into hydrophobic entities by using cationic materials such as lipids, polyethyleneimines (PEI), polyamino acids, polyvinyl pyrrolidone (PVP) etc. A more appropriate material is cationic lipids, which with their hydrophobic carbon chains confer greater hydrophobic characteristics to the nucleic acids. The nucleic acids are carefully titrated against these cationic materials to make them hydrophobic. For example, we use the cationic lipid, 1,2-dioleoyl-3-Trimethylammonium-Propane (DOTAP), to convert siRNA into a hydrophobic entity, or a hydrophobic ion-pair complex (HIP) of siRNA-DOTAP. This is done by the Bligh-Dyer method wherein the siRNA is dissolved into an aqueous phase (water or buffers), and the DOTAP is dissolved into an organic solvent such as dichloromethane or chloroform (Reimer et al. (1995) Biochemistry 341 12877-12883; Wong et al. (1996) Biochemistry 35, 5756-5763). The two solutions are mixed with an alcohol such as methanol or ethanol in the ratio of 1:2.1:1 (siRNA:methanol:DOTAP). The siRNA and DOTAP are used at equimolar concentrations. This leads to the formation of a monophase, which upon mixing for some time, 15 minutes to more than 3 hours, leads to the formation of the HIP complex. The complex is formed due to the interaction between the phosphate groups on the nucleic acids and the cationic amines on the DOTAP. Upon nullification of the charges, the entire entity becomes hydrophobic with the long carbon chains of DOTAP allowing for greater solubility in organic solvents. After mixing, the monophase is separated into an organic phase and an aqueous phase by addition of equal volumes of the organic solvent and water or buffer (final ratios: (2:2.1:2)). The HIP can then be extracted from the organic phase and either dried or used as is. Greater than 99% of siRNA can be extracted into the organic phase by this method.

The HIP is then solubilized into an appropriate organic solvent such as chloroform along with PLGA-PEG. For example, PLGA-PEG at concentrations ranging from 10-100 mg/ml is dissolved in chloroform along with the HIP. The HIP concentration can be varied from 0 to 10% of the total polymeric concentration. The solution is homogenous and added dropwise into a bath of non-solvents, i.e. solvents that precipitate PLGA. In this study, ethanol was used to form nanoparticles at ethanol:chloroform volume ratios ranging from 1:1 to 100:1 and preferably at a ratio of 2:1. Upon contact with ethanol, the chloroform in the droplet moves into the bulk thus causing the PLGA to precipitate and form nanoparticles. Sometimes additives such as non-ionic surfactants like Pluronic F-68 (PF68), Pluoronic F-127 (PF127), PVA, Tween etc. can be used to prevent agglomeration of the nanoparticles. The PEG is soluble in ethanol and hence starts to move into the ethanol bath along with chloroform. Hence, by the time PLGA precipitates into nanospheres, the PEG localizes to the surface thus providing a hydrophilic coating. Thus, the nanoparticles have a PLGA core and a PEG coating. The free end of PEG can carry different functional groups as mentioned above, thus providing a platform for surface modification of the nanoparticles with ligands, antibodies, aptamers etc.

The nanoparticles that are present in the organic solution of ethanol and chloroform can be obtained by addition of excess and equal volumes of ethanol and water. For example, for a nanoparticle solution of 50 mg, one can add 25 ml of ethanol and water each to the organic solution. The organic solvents in this mixture are then evaporated using either rotary evaporator at not more than 40° C., or by stirring for long periods of time. The ethanol and water can contain additives such as PF-68 or Tween or PVA to aid in nanoparticle recovery and preventing agglomeration. The nanoparticles are then recovered in the water phase, which is then centrifuged for 10-45 minutes to recover the nanoparticle pellet. The typical speeds used are 10000×g which can be varied appropriately. The nanoparticles can also be recovered using various filtration setups with appropriate membrane cutoffs. The use of filtration exposes the nanoparticles to lower centrifugal forces of say 3000×g thus reducing the chances of particle agglomeration. This process is usually carried out 2-3 times to wash the nanoparticles of impurities. Ethanol can also be mixed with water or buffers in varying ratios, wherein addition of the polymer/HIP solution of chloroform will yield an emulsion. The chloroform and ethanol in this emulsion can then be evaporated under vacuum at elevated temperatures to yield nanoparticles encapsulating siRNA.

The advantage of PLGA-based nanoparticles is that they can be stored in a dry form for long periods of time. Nanoparticles tend to agglomerate during the drying step, wherein frozen solutions are lyophilized under high vacuum at −80° C. To prevent agglomeration, various additives are used such as sucrose, D-mannitol, Sorbitol, dextrose, trehalose, albumin protein or PVA etc. The nanoparticle pellet recovered from the centrifugation or filtration process is redispersed into water containing 10% sucrose or 4% sucrose+3% mannitol+PVA at 10-20% of the nanoparticle weight. The nanoparticle solution is then frozen in liquid nitrogen and lyophilized for 2-3 days. The dried nanoparticle powder can then be reconstituted into water or an appropriate buffer for further use.

A potential modification to the above process involves the addition of a cationic entity between PEG and PLGA. The presence of short cationic molecules (Grey circle, FIG. 9) will enhance the escape capabilities of the nanoparticle from the endosome. As the nanoparticle is formed, the cationic chains will be localized at the nanoparticle surface along. This is possible via the ‘proton sponge’ effect wherein the lower pKa of the cationic species acts as a sponge for protons that enter the endosome via ATP dependent pathways. The buffering effect of these cationic entities will increase the osmotic pressure inside the endosome leading to its rupture. Another possibility is the use of sheddable PEG coatings, which upon entry into the endosome can release the PEG coating, thus exposing the surface of the nanoparticles to the endosomal environment. Thus, careful selection of terminating groups on the nanoparticle surface such as amines will cause the rupture of the endosomes due to the aforementioned proton sponge effect. Use of carboxylic acid terminating groups on the surface can lead to another mechanism of endosomal escape, as described by Panyam et al. (2002) Faseb J 16, 1217-1226 wherein the -carboxylic acid has a pKa of ˜4.5. As the pH in the endosome is lowered, the —COO⁻ gets protonated to —COOH and becomes hydrophobic. This hydrophobic surface fuses with the endosomal membrane and causes its rupture and release of the contents into the cytoplasm.

In an example, we attached galactose to the surface of the nanoparticles. There are two ways to attach ligands to the nanoparticle surface: 1) Attach the ligand to the free PEG end in the PLGA-PEG copolymer, as described by Gu et al. (2008) PNAS 105, 2586-2591, 2) attach the ligand after formation of the nanoparticles by standard coupling chemistries. We chose the first method wherein lactobionic acid was coupled to an amine terminating PLGA-PEG-NH₂. In order to obtain PLGA-PEG-NH₂, we followed the same approach as described above. To generate an amine terminating PEG-PLGA, a diamine-PEG was used at a 300-500% molar excess to PLGA in the reaction. Lactobionic acid was coupled to the amine group on the PEG by dissolving the reactants in DMSO with the addition of EDC, NHS ester and DIEA. The reaction was allowed to proceed for at least 6 hours. The reaction mixture was then added to cold ethanol to precipitate the copolymer and separated by centrifugation. The copolymer was washed 2-3 times by repeating the same procedure. Afterwards, the copolymer was dried under vacuum and stored. Upon nanoparticle fabrication, the galactose ligand gets localized on the surface. The presence of galactose was assayed and quantified by using the Amplex Red Galactose Assay (Invitrogen).

The encapsulation efficiency of siRNA into nanoparticles was determined by measuring the siRNA not encapsulated, present in the ethanol bath after nanoprecipitation. Mass balance of siRNA yielded the siRNA encapsulated inside nanoparticles. Sybr Gold (invitrogen) was used to quantify siRNA.

In order to investigate the release of siRNA from the nanoparticles, known amount of the nanoparticle was dispersed into PBS (pH 7.4) and placed in an incubator with gentle shaking. The amount of siRNA released into PBS was quantified using Sybr Gold. The siRNA that was released from the nanoparticles was also characterized for degradation and activity. Gel electrophoresis was used to determine degradation of the siRNA, whereas HepG2-CBL cells were used to investigate siRNA activity. HepG2-CBL are human hepatocellular carcinoma cells that have been stably transfected with a Click Beetle Luciferase (CBL) reporter gene. The activity of siRNA released from the nanoparticles was compared to the original siRNA by introducing them into HepG2-CBL using commercially available Lipofectamine RNAiMax (Invitrogen). The siRNA was targeted against CBL.

Uptake of nanoparticles prepared from PLGA-TEPA-PEG polymer with and without galactose as a targeting ligand:

Nanoparticles without galactose—PLGA from Boehringer Ingelheim (RG503H) RG503H-TEPA-PEG-CH3 (35 mg)+Hydrophobic Phospholipid (TRITC DHPE P-1391, Invitrogen) (125 μg) was dissolved in 1.8 ml of Acetone and added into a stirring Water solution (6 ml). The mixing was continued overnight in the cold room at 4° C. to remove the acetone. This was done to prevent evaporation of the water. The mixture was centrifuged and the nanoparticles were collected and weighed.

Nanoparticles with galactose—RGS03H-TEPA-PEG-CH₃ (22.5 mg) RG503H-PEG-Gal (12.5 mg)+Hydrophobic Phospholipid (TRITC DHPE P-1391, Invitrogen) (125 μg) was dissolved in 1.8 ml of Acetone and added into a stirring Water solution (6 ml). The mixing was continued overnight in the cold room at 4° C. to remove the acetone. This was done to prevent evaporation of the water. The mixture was centrifuged and the nanoparticles were collected and weighed.

______TRITC was used in this process because this dye is a phospholipid and has lower solubility in polar solvents such as water. Hence, greater encapsulation of the dye is possible in the nanoparticles. The Eₓ is 540-550 and Eₘ is 575.

Cell Uptake Studies: HepG2 cells were plated at a concentration of 300,000 cells/well (6-well plate) 48 hours prior to experiments. The cells were rinsed with PBS once very carefully, as the cells tend to come off the plate upon washing. The cells were then incubated in 1 ml of OptiMEM serum free media for 2 hours. Nanoparticle solutions were prepared in the OptiMEM media at two different concentrations (0.26 and 0.78 mg/ml) and were added to the wells. The nanoparticles were incubated for about 1.5 hours at 37° C. and removed. The cells were then removed from the plate using PBS (trypsin is not required as the cells come off easily) and the cell/PBS solution was centrifuged at 800 rpm in the microcentrifuge. The PBS was removed and the pellets were redispersed in the cell culture media (DMEM+FBS) and plated again in the 6-well plates overnight.

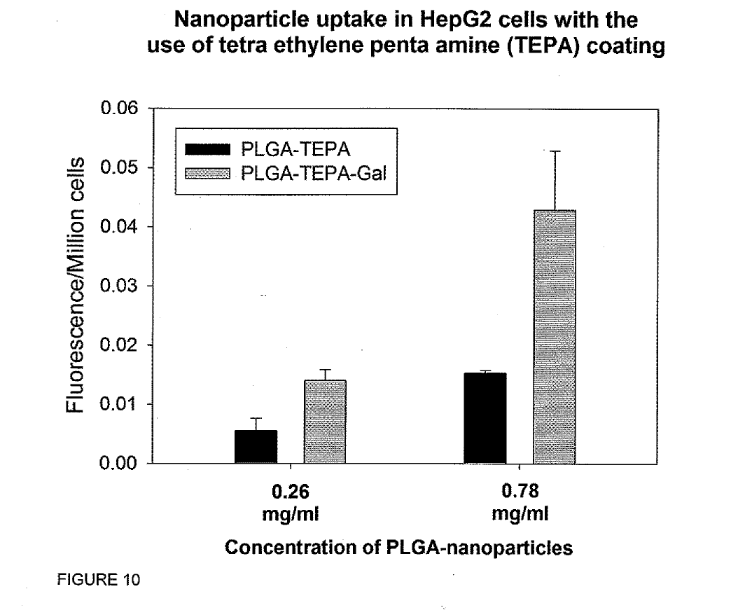

The next day the cells were removed from the plate and washed 2 times with PBS and redispersed into 500 μl of PBS. The concentration of each group of cells was determined with the Nexcelom cell counter. 20 μl of the cell solution with 30 μl of PBS was added to each well in a black 96-well microplate and the fluorescence was measured at Eₓ is 540 and Eₘ is 575. The results are displayed in FIG. 10 wherein the fluorescence measurements were normalized to the number of cells. The results clearly indicate that the nanoparticles with galactose on the surface exhibit higher uptake into the HepG2 cells. The presence of TEPA inserted between PLGA and PEG does not reduce the uptake of nanoparticles into cells, and may probably enhance the uptake. The presence of amine rich moieties with pKa of 6.0 and lower can act as proton sponge in the endosome. With a decrease in the pH of the endosome, there is an excess inflow of H+ ions that end up protonating the amine groups, thus contributing to an increase in the osmotic pressure in the endosome. The increase in osmotic pressure leads to subsequent rupture of the endosome, thus emptying the nanoparticles into the cytoplasm. TEPA exhibits different pKa values for the different amine groups (see Proc. Natl. Acad. Sci. USA, Vol. 93, pp. 1585-1590, February 1996, Biochemistry), thus contributing to the proton sponge effect in the endosome.

The nanoparticles were also tested for delivery of CBL-siRNA across cell membrane using the HepG2-CBL cell line, and these nanoparticles had galactose on the surface. Nanoparticles that encapsulated control/mock siRNA, decorated by galactose on the surface, were used as a control in these studies. The HepG2-CBL cells were incubated in serum-free OptiMem media for at least 2 hours to reorient the Asioglycoprotein receptors (AsGPR) for accessible conformation and to achieve maximum galactose uptake potential. The nanoparticles were dispersed into OptiMem at concentrations ranging from 0.1 to 1 mg/ml and added to the cells, and incubated for 3 hours. Bioluminescence signal from the CBL reporter in the cells were recorded, and siRNA targeting towards the mRNA of the CBL gene was observed as a % reduction in bioluminescence.

The pharmacokinetics of nanoparticles in living subjects were studied by using an oligonucleotide that had been conjugated to a near infra red (NIR) dye, Licor 800CW (Licor). The oligonucleotide was the same length as a siRNA and could be used as a model nucleic acid payload for the nanoparticles. The oligos with Licor800CW were subjected to the same process for manufacture of nanoparticles as described above. Known amounts of nanoparticles with galactose, carrying the oligo-Licor were injected into Balb/C female mice. The mice were then imaged with the ART eXploreOptix fluorescence imaging system to study the biodistribution of the nanoparticles after 24 hours. The mice were then sacrificed and various organs were harvested. The fluorescence from each organ was measured to quantify the amount of nanoparticles present in each organ. In order to test efficacy of the nanoparticles in living subjects, we investigated the knockdown of the CBL gene in a transgenic animal model. Transgenic C57B6 mice were obtained wherein a β-actin promoter controlled the expression of CBL. The mice were engineered to express only in the liver, and hence provided an excellent model to test liver delivery of siRNA. Nanoparticles decorated with galactose, and carrying siRNA targeted towards CBL or a control siRNA were introduced into the mice intravenously at concentrations of 2.5 mg siRNA/kg mouse. The mice were then imaged for bioluminescence signal from the liver over a period of 7 days. The bioluminescence was compared to the original signal from the mice and reduction in the signal was considered as a result of degradation of CBL mRNA due to deliver of the siRNA from the nanoparticles.

What is claimed is:

1. A method of generating polymer-encapsulated nanoparticles of a nucleic acid, the method comprising:
titrating the nucleic acid against a cationic amphipathic molecule to create a hydrophobic ion-pair (HIP);
solubilizing the HIP and a polymer in an organic solvent to provide a mixture;
mixing the mixture into a miscible, or non-miscible, non-solvent with stirring;
wherein the nucleic acid is precipitated in encapsulated, nanosized particles.

2. The method of claim 1, wherein the nanosized particles are from 10 nm to 10 μm in diameter.

3. The method of claim 1, wherein the nucleic acid is RNA.

5. The method of claim 1, wherein the nucleic acid is DNA.

6. The method of claim 1, wherein the polymer is a biodegradable polymer.

7. The method of claim 6, wherein the biodegradable polymer is a conjugate of PLA-PEG; PLGA-PEG, PLG-PEG, or a mixture thereof.

8. The method of claim 7; wherein the conjugate comprises an amine rich polymer inserted between the PEG moiety and the PLA, PLGA or PLG moiety.

9. The method of claim 8 wherein the amine rich polymer is selected from diethylene triamine (DETA), tetraethylene pentaamine (TEPA), ethylene diamine (EDA), tetraethylene tetraamine (TETA), bis(hexamethylene triamine) (BHMT) and pentaethylene hexamine (PEHA).

10. The method of claim 7, wherein the conjugate comprises a cationic entity between PEG and the polymer.

11. The method of claim 7, wherein the PEG is conjugated through a sheddable linker.

12. The method of claim 1 wherein the cationic amphipathic molecule is selected from DOTIM; DDAB; DOTMA; DOTAP; DMRIE; EDMPC; DCChol; DOGS; and MBOP.

13. The method of claim 12 wherein the cationic amphipathic molecule is DOTAP.

14. The method of claim 1, wherein the nucleic acid is solubilized at a concentration from about 0.001 mg/ml to about 10 mg/ml.

15. The method of claim 14, where the ratio of compound to polymer as a weight percentage is from about 1:1000 to about 1:5.

16. A population of polymer-encapsulated nanoparticles of a nucleic acid produced by the method according to claim 1.

17. The population of polymer-encapsulated nanoparticles of claim 16, further comprising a pharmaceutically acceptable excipient.
